(19) United States
(12) Patent Application Publication
(10) Pub. No.: US 2021/0138217 A1
(43) Pub. Date: May 13, 2021

(54) DEVICE FOR IMPLANTING OBJECTS UNDER THE SKIN

(71) Applicant: H&B ELECTRONIC GMBH & CO. KG, Deckenpfronn (DE)

(72) Inventors: Tobias MORLOK, Mötzingen (DE); Dariusz PETRY, Aidlingen (DE)

(73) Assignee: H&B ELECTRONIC GMBH & CO. KG, Deckenpfronn (DE)

(21) Appl. No.: 16/635,770

(22) PCT Filed: Jun. 26, 2018
(86) PCT No.: PCT/EP2018/067123

§ 371 Date: Jan. 31, 2020

(30) Foreign Application Priority Data
Aug. 23, 2017 (DE) 20 2017 105 048.3

(51) Int. Cl. A61M 37/00

(52) U.S. Cl. CPC A61M 37/0069

(57) ABSTRACT

An implanting device for positioning an object under a patient's skin includes a housing, a displaceable cannula holder, a cannula for receiving the object to be implanted and a tappet, which can be fixed relative to the housing. The cannula is displaceable from a first piercing position into a first operating position in order to deposit a first object, and the cannula is displaceable jointly with the tappet into a second piercing position and into a second operating position in order to deposit a second object. In order to move into the second piercing position, the cannula holder can be fixed to a first housing part, which is fixedly connected to the distal end of the tappet and is held displaceably on a second housing part forming the housing outlet.

The invention relates to an implantation device for placing at least one object under a patient's skin according to the preamble of claim 1. This implantation device has a housing and a cannula holder, which is held so as to be displaceable relative to the housing and to which a cannula can be attached or is attached, said cannula having a distal tip and a cavity for receiving the at least one object to be implanted. The implantation device further has a tappet which can be fixed or is fixed relative to the housing and forms a distal end which can be positioned in the cavity of the cannula to support in the proximal direction the objects to be implanted. The cannula can be displaced from a first insertion position relative to a housing outlet via a first return stroke path along the tappet into a first operating position, in order to thereby deposit a first object received in the cavity. The cannula is displaceable relative to the housing outlet together with the tappet, that is to say in a positionally stable manner relative to the tappet, from the first operating position into a second insertion position. In addition, the cannula is displaceable from the second insertion position via a second return stroke path along the tappet into a second operating position, in order to deposit or place a second object received in the cavity of the cannula. In this way, the cannula can be brought, together with the tappet, into a position from which at least a second predefined placing movement can be carried out to deposit another object. As a result, the implantation device can be reloaded or wound up in order to be able to securely place, by means of a specified relative movement of the cannula with respect to the tappet, at least two implants received one behind the other in the cannula.

Such implantation devices enable the placement of, in particular, elongate implants, which are used, for example, for the long-term release of medicinal active substances or hormones in the body of a human or an animal. Alternatively, the implants can also be formed by electronic components which are used, for example, to identify the wearer or to supply energy to medical implants.

DE3880676T2 shows an implantation device for arranging two pharmaceutical implants in a fan-shaped manner under a patient's skin. For this purpose, the two implants are arranged one behind the other in a cannula, which is held on a hub. To place the first implant, the hub with the cannula is slid along a tappet and then moved again in the opposite direction in preparation for the second placement procedure. A telescopic part of the tappet is extended, on which the second implant is supported at its rear during the second placement procedure.

DE69731843T2 describes an implantation device for placing several solid bodies. For this purpose, the solid bodies are arranged one behind the other in a cannula, wherein the rearmost solid body is supported at its rear by a tappet that can be displaced along the cannula. Detent means are provided on the tappet that indicate to the user suitable positions of the tappet for placing the individual solid bodies when the cannula is moved.

US2003/0040699A1 shows an implantation device for carrying out a single placement procedure, in which a cannula filled with implants is moved along a tappet by actuating a sliding button, as a result of which the implants are released.

US2004/0199140, like US2008/0221510A1 and US2009/0012463A1, shows an implantation device having a tappet fixedly mounted on the housing, in which the cannula is connected to a sliding button that can be displaced along a curved guide. The sliding button allows the cannula to be moved along the tappet to release the implants received in the cannula.

WO 2006/077250 A1 discloses an implantation device for placing elongate implants under the skin of humans or animals. This device has a cannula protruding from a housing, in which the implant is supported at a proximal end on a tappet which is held in a positionally stable manner in the housing. After the insertion and positioning of a distal section of the cannula under the patient's skin, the cannula is slid in the proximal direction of the housing by means of a sliding handgrip, whereby the implant is placed under the patient's skin.

A disadvantage of the known implantation device is that it can only be used to carry out a single placing movement that is predefined in terms of its length, which is why in each case only one safe placement procedure for positioning an elongate implant under a patient's skin can be carried out. In contrast, before a second object can be placed with the same implantation device, both the cannula and the tappet must be replaced or at least cleaned, and the further object must be re-inserted. In addition, due to the relatively long actuation path of the handgrip, the implantation device can generally only be operated with two hands.

The object of the invention is to avoid the mentioned disadvantages in a generic implantation device and to enable both convenient and trouble-free implantation, in particular of two or more objects.

This object is achieved by an implantation device having the features of claim 1. For displacement into the second insertion position, the cannula holder can be fixed to a first housing part, which is fixedly connected to the distal end of the tappet. This first housing part is held displaceably on a second housing part, which in turn forms the housing outlet. As a result, the implantation device can be reloaded for the second placement procedure by a relative movement between the two housing parts which is easy to carry out and ensures a particularly convenient performance of the at least one further placement procedure.

In this case, it is advantageous if the first housing part is slidably mounted on the second housing part in order to ensure a trouble-free and precise relative movement between the two housing parts.

Advantageously, the second housing part has anti-slip means on an outer side thereof. As a result, it is possible, by only gently pressing the second housing part against the skin of the patient in question, to hold said housing part in a positionally stable manner while the first housing part is displaced relative thereto. In this way, after the first implant has been placed, the implantation device can be brought with only one hand into the second insertion position, from which the second placement procedure can be carried out.

In addition, it is advantageous if, between the first housing part and the second housing part, housing locking means are provided which securely fix the two housing parts onto each other until the first insertion position of the cannula is reached. This can ensure that there is no troublesome relative movement between the two housing parts during the first placement procedure.

In a particularly advantageous embodiment of the implantation device, return means are provided, by means of which the displacement of the cannula over at least one of the return stroke paths can be carried out automatically. In this way, the cannula can be displaced automatically to place the implant in question, which enables particularly convenient handling of the implantation device. In particular, this enables convenient one-handed operation of the implantation device.

Furthermore, it is advantageous if the return means have a mechanical energy store, by means of which the cannula holder or the cannula can be displaced over the relevant return stroke path. For this purpose, the energy store can be connected to the cannula holder by, for example, a rolled spring. In this way, the return means required for the automatic return movement can be made available in a particularly simple and cost-effective manner.

In a further advantageous embodiment, the return means have a gear mechanism, by which the cannula holder can be displaced. In this way, a preferred transmission between the movement of the mechanical energy store and of the cannula holder can be set.

It is also advantageous if the return means have a damping device, by means of which a displacement speed during the movement over at least one of the return stroke paths can be set. In this way, the displacement speed of the cannula during its movement over the return stroke path can be limited in such a way that minimal impairment of the tissue in the implantation area can be ensured.

It is advantageous if the return means have a trigger button, by means of which the displacement over at least one of the return stroke paths can be triggered. The trigger button can in particular be designed in such a way that an automatic stop occurs after a displacement over the specified return stroke path, irrespective of the position of the trigger button. In addition, it can be provided that, after passing through the first return stroke path, the trigger button comes back into engagement with the cannula holder, which is displaced over a second return stroke path after the trigger button has been pressed again. In any case, such a trigger button enables particularly convenient operation of the implantation device with only one hand.

In addition, it is advantageous if the trigger means is provided with fixing means that can be releasably connected to complementary fixing means of the cannula holder. This makes it possible to use the trigger button for at least two successive placement procedures, despite its position on the housing remaining the same.

In an alternative embodiment of the implantation device, the displacement of the cannula or the cannula holder over at least one of the return stroke paths can be carried out manually, in which case the implanting person can control the movement of the cannula over the relevant return stroke path particularly well.

For this purpose, it is advantageous if, for manual displacement of the cannula or the cannula holder, a sliding button is provided, which is slidable on the housing and can be released from a lock by applying force. This releasable lock means that an inadvertent displacement of the sliding button and an associated unintended actuation of the cannula can be avoided.

Advantageously, in a final operating position, which corresponds, for example, to the second operating position, the cannula is received completely within the housing. In this way, injuries caused by the cannula protruding from the housing can be prevented after all the intended placement procedures have been completed.

In this case, it is advantageous if an end position safety device is provided, by means of which the cannula holder can be fixed in said final operating position relative to the housing, so that an inadvertent displacement of the cannula holder or the cannula out of the housing outlet can be avoided.

The tappet can advantageously be separated from the housing in the second operating position. This makes it possible, for example, to separate and dispose of the cannula, and if appropriate also the tappet, from the implantation device after the placement procedures have been carried out.

In a particularly preferred embodiment, the cannula and the tappet each have a cross-section which deviates from a circular profile, and which can be, for example, polygonal, oval, kidney-shaped, elliptical or the like. This makes it possible to receive a correspondingly shaped object in a specified rotational position with respect to the longitudinal axis of the cannula, to guide said object along the cannula during the placement procedure and to position it accordingly under the patient's skin. In this way, the implant can assume a position that is particularly advantageous with regard to a specific function of the implant. For example, a side of the implant that is sensitive to light or utilises light energy can be oriented towards the skin surface in order to ensure optimum energy generation.

Advantageously, the housing forms a stop at a distal end, which stop is used to limit an insertion depth. Such a stop makes it particularly easy for the user of the implantation device to maintain an insertion depth of the cannula with respect to the patient's skin that is suitable for the implantation procedure.

In this case, it is advantageous if a contact surface is formed on the stop and is bevelled at a specified insertion angle relative to a longitudinal extension of the implantation device. Such a contact surface allows not only a suitable insertion depth but also a preferred insertion angle with respect to the patient's skin to be maintained in a particularly simple manner.

In addition, a viewing area is advantageously provided on the housing, which can be formed, for example, by a recess, an indentation or a transparent section or a window of the housing, and through which a section of the cannula protruding from the housing outlet or a length portion mark on the cannula can be seen.

In a preferred embodiment of the implantation device, moreover, the parts that come into contact with tissue of the patient during a placement procedure and are thereby contaminated are at least partially exchangeable to enable reuse of the rest of the implantation device while minimising the need for cleaning. For this purpose, the cannula or the cannula together with the tappet is held exchangeably on the implantation device. Moreover, the cannula holder or even the entire second housing part with the cannula can also be at least partially designed as a single-use device which is fixed exchangeably on the first housing part.

In any case, it is advantageous if the exchangeable elements can be fixed to the rest of the device via a safety coupling, and this safety coupling can only be released when the final operating position has been reached. In this way, the exchangeable parts can be prevented from being inadvertently released before the respective placement procedures have been completed.

It is also advantageous if, for reuse of the implantation device with a new interchangeable element, the mechanical energy store can be pretensioned by manual relative movement of a housing section, wherein a safety catch is provided which in an initial position blocks the relative movement of the housing section and can be displaced into a release position in which the relative movement is released. This makes it possible to design the implantation device in such a way that its mechanical energy store can be reloaded at any time, thereby avoiding inadvertent loading of the implantation device and a risk of injury resulting therefrom.

The at least one object to be implanted is advantageously pre-loaded in the cannula, so that the implantation device is ready for operation.

In this case, it is advantageous if the received object is arranged at the distal tip of the cannula, whereby the implant itself is used to close the tip. This can prevent or at least reduce undesired entry of tissue into the cannula.

Furthermore, the above-mentioned object is achieved by an implantation arrangement comprising an implantation device in one of the above-mentioned embodiments, the implantation arrangement having a separate object to be implanted, which can be inserted into the cannula. This makes the implantation device available in the form of a partially reusable, coordinated set.

In this case, it is advantageous if an insertion aid is provided for inserting the at least one object into the cavity and which can be attached to one end of the cannula. Such an insertion aid, which can for example be funnel-shaped, enables simplified insertion of implants into even relatively fine cannulas.

In addition, it is advantageous if the at least one object is received in a sleeve which is inserted into the cannula together with the object. Such a sleeve enables in particular a particularly convenient insertion of two or more implants into the cavity of the cannula.

It is noted that all of the features of the subject matter according to the invention described above are exchangeable or combinable with one another, provided that an exchange or combination thereof is not ruled out for technical reasons.

An exemplary embodiment of the invention is shown in the Figures, in which:

FIG. 1 is a perspective view of an implantation device according to the invention in a first insertion position,

FIG. 2 is a longitudinal section through the implantation device according to FIG. 1,

FIG. 2A is a perspective view of an arrangement of fixing means and complementary fixing means of the implantation device according to FIG. 1,

FIG. 3 is a longitudinal section through the implantation device according to FIG. 1 in a first operating position,

FIG. 4 is a longitudinal section through the implantation device according to FIG. 1 in a second insertion position,

FIG. 5 is a longitudinal section through the implantation device according to FIG. 1 in a second operating position,

FIG. 6 is a longitudinal section through the implantation device according to FIG. 1 in the second operating position and additionally displaced tappet,

FIG. 7 is a longitudinal section through the implantation device according to FIG. 1 after all placement procedures have been completed and the housing has been closed,

FIG. 8 is a view of the implantation device with an alternative embodiment of the housing,

FIG. 9 is a perspective view of the implantation device according to FIG. 8 when inserted into a skin layer,

FIG. 10 is a view of the implantation device according to FIG. 8 when an insertion depth stop is in contact with the skin layer,

FIG. 11 is a view of the implantation device according to FIG. 8 when an insertion site of the skin is in contact with a second housing part,

FIG. 12 is a view of an implantation device according to the invention in the first insertion position and inserted into the skin layer,

FIG. 13 is a view of the implantation device according to FIG. 12 with the second housing part in contact with a surface of the skin layer,

FIG. 14 is a view of the implantation device according to FIG. 12 after a first placement procedure,

FIG. 15 is a view of the implantation device according to FIG. 12 after pivoting about a horizontal angle,

FIG. 16 is a view of the implantation device according to FIG. 12 in a second insertion position,

FIG. 17 is a view of the implantation device according to FIG. 12 after a second placement procedure,

FIG. 18 is a perspective view of the implantation device with a further alternative embodiment of the housing and with the second housing part removed,

FIG. 19 is a longitudinal section through a first housing part of the implantation device according to FIG. 18,

FIG. 20 is a longitudinal section through a second housing part of the implantation device according to FIG. 18,

FIG. 21 is a perspective view of an alternative embodiment of the implantation device,

FIG. 22 is a longitudinal section through the implantation device according to FIG. 21 in the first insertion position,

FIG. 23 is a longitudinal section through the implantation device according to FIG. 21 in the first operating position,

FIG. 24 is a longitudinal section through the implantation device according to FIG. 21 in the second insertion position,

FIG. 25 is a longitudinal section through the implantation device according to FIG. 21 before the second placement procedure,

FIG. 26 is a longitudinal section through the implantation device according to FIG. 21 in the second operating position,

FIG. 27 is a longitudinal section through housing locking means of the implantation device in the first operating position of the cannula holder,

FIG. 28 is a perspective view of a cannula of the implantation device according to the invention with attached insertion aid,

FIG. 29 is an exploded perspective view of the cannula with a sleeve which can be inserted therein and two objects to be implanted,

FIG. 30 is a perspective view of the cannula with a profile which deviates from a circular profile, and

FIG. 31 is an enlarged view of a distal tip of the cannula according to FIG. 30.

FIGS. 1 and 2 show an implantation device 2 for placing two objects 4A, 4B in the exemplary form of elongated hormone implants, which can be implanted in a patient for long-term delivery of active substances for contraception. Alternatively, the objects 4A, 4B can also be formed as implants for delivering other medications for humans or animals or can contain electronic elements which, for example, enable identification of the person concerned for access control or for payment services or which serve as solar cells as part of a medical or other electronic implant.

In any case, for the placement procedure the objects 4A, 4B to be implanted are received in a cavity 8 of a cannula 10 of the implantation device 2. Here, the first object 4A to be placed is positioned at a distal tip 12 of the cannula 10 in such a way that as far as possible it closes the opening of said cannula.

In the position shown in FIGS. 1 and 2, the cannula 10 is in a first insertion position, in which it protrudes from a housing outlet 14 of a housing 16 of the implantation device 2. The cannula 10 has a first length portion mark M1 and a second length portion mark M2, both of which are, in the first insertion position, disposed outside the housing 16.

The housing 16 of the implantation device 2 has a first housing part 18, which is slidably held on a second housing part 20 which forms the housing outlet 14.

As can be seen from FIG. 2, the cannula 10 is fastened to a cannula holder 22, which can be displaced in a guided manner within the housing 16. In the illustrated first insertion position, the cannula holder 22 is held by fixing means 24 of a trigger button 26, which cooperate with first complementary fixing means 25A of the cannula holder 22. The interlocking fixing means 24 and complementary fixing means 25a hold the cannula holder 22 in a distal position within the housing 16 against the pretension of a rolled spring 28 of a mechanical energy store 30 acting on the cannula holder 22. The energy store 30, the rolled spring 28 and the release button 26 form return means for automatic displacement of the cannula holder 22 in the proximal direction.

The implantation device 2 further comprises a tappet 32, which is fixedly connected to the first housing part 18 and protrudes into the cavity 8 of the cannula 10. A distal end 34 of the tappet 32 supports the second object 4B to be implanted in the proximal direction.

By actuating the trigger button 26, the fixing means 24 can be released from the first complementary fixing means 25A of the cannula holder 22, whereby the latter is displaced, due to the pretension by the rolled spring 28, in the proximal direction, to a stop position in which the fixing means 24 of the trigger button come into engagement with second complementary fixing means 25B of the cannula holder 22, as shown in particular in FIG. 2A. At the same time, the cannula 10 is thereby displaced from the first insertion position over a first return stroke path along the tappet 32 through the housing outlet 14 into a first operating position, shown in FIG. 3. As can be seen from FIG. 3, this movement releases the first object 4A, while the second object 4B, supported by the tappet 32, is displaced to the distal tip 12 of the cannula.

From this first operating position shown in FIG. 3, the first housing part 18 can now be displaced in the distal direction with respect to the second housing part 20 by applying manual force. As a result of the cannula holder 22 being fixed to the trigger button 26 via the fixing means 24 and complementary fixing means 25B, the cannula 10 is also displaced relative to the second housing part 20 and thereby moves from the first operating position together with the tappet 32 into a second insertion position, shown in FIG. 4.

In this position shown in FIG. 4, the fixing means 24 can be completely separated from the fixing counter-means 25B of the cannula holder 22 by actuating the trigger button 26 again, whereby the cannula holder is displaced as a result of the further pretension by the rolled spring 28 in the proximal direction to an end stop position. In this end stop position shown in FIG. 5, the cannula holder 22 forms, together with the first housing part 18, an end position safety device 36, which is formed, for example, by a snap connection and permanently secures the cannula holder 22 in the end stop position. At the same time, through the displacement of the cannula holder 22, the cannula 10 is displaced from the second insertion position, over a second return stroke path along the tappet 32 and into the housing outlet 14, into a second insertion position shown in FIG. 5. The second object 4B is now also released by this movement, while the cannula itself is received completely within the second housing part 20.

In order to ensure a suitable speed and uniform displacement of the cannula holder 22 into at least one of the operating positions during the automatic movement generated by the return means, a gear mechanism 37 acting between the energy store 30 and the cannula holder 22 and/or a damping device 39 can also be provided, as in each case represented by broken lines in FIG. 2.

After completion of the last placement procedure, a further displacement of the cannula holder 22 in the proximal direction as a result of the mechanical energy store 30 can also be provided, by means of which the tappet 32 is separated from the first housing part 18 and can be displaced completely into the second housing part 20, as shown in FIG. 6.

The first housing part 18 can then be slid in the proximal direction relative to the second housing part 20 by manual application of force, in order to be able to close the housing 16 with the cannula 10 received completely therein and the completely received tappet 32, as shown in FIG. 7, and, if appropriate, to dispose of it.

FIG. 8 shows the implantation device 2 with an alternative embodiment of the housing 16. This has a second housing part 20, which at a distal end 40 is shortened compared to the first housing part 18, and a viewing area 38. The first housing part 18 forms a stop 42 at the distal end 40, which stop, as can be seen from FIGS. 9 and 10, limits an insertion depth T of the cannula 10. The viewing area 38, which is formed by an indentation, enables at the same time good visibility of a cannula section or the length portion mark M1.

As shown in FIG. 10, the stop 42 is formed by a contact surface 44, which is bevelled at a specified insertion angle WI with respect to a longitudinal extension of the implantation device 2.

As can be seen from FIG. 11, due to the shortened design of the second housing part 20 compared to the first housing part 18, the distal end of the second housing part 20 can also serve as an additional end stop with which an insertion site E comes into contact as soon as the cannula 10 is inserted into a skin layer 6 up to the second length portion mark M2, as shown in FIG. 11.

The placement of the objects 4A, 4B to be implanted is described below with reference to the design variant of the implantation device 2 as in FIGS. 8 to 11:

To place the first object 4A, as can be seen from FIG. 12, the cannula 10, protruding from the housing 16, is first inserted into the skin layer 6 of the patient up to the first length portion mark M1, with the contact surface 44 acting as a stop 42 to limit the insertion depth. The insertion is carried out, as shown, at the insertion angle WI.

The implantation device 2 is then pivoted around the insertion site E in such a way that the second housing part 20, as shown in FIG. 13, rests on the surface of the skin layer 6. Thereupon, the implantation device 2 is displaced in the direction of the insertion site E until the cannula 10 is displaced into the insertion site E up to the second length portion mark M2 or until the insertion site E lies against the distal end of the second housing part 20.

By actuating the trigger button 22, the cannula 10 is displaced over the first return stroke path into the second housing part 20 and into the first operating position as shown in FIG. 14. The first object 4A remains, as shown, under the skin layer 6 at the insertion site E.

To then place the second object 4B, the implantation device 2 is rotated around the insertion site E on the surface of the skin layer 6 over a horizontal angle WH, as shown in FIG. 15.

The first housing part 18 is then slid relative to the second housing part 20 in the direction of the insertion site E by manual application of force. In order to be able to ensure a stable position of the second housing part 20, anti-slip means 45 are provided on its surface, and can be formed, for example, by a rubber coating or by knurls.

Due to the relative movement between the two housing parts 18, 20, the cannula 10 moves from the first operating position into the second insertion position relative to the second housing part 20, and is displaced under the skin layer 6 until the insertion site E again lies against the second housing part 20, as can be seen from FIG. 16.

By actuating the trigger button 22 again, the cannula 10 is displaced out of the second insertion position along the second return stroke path into the second operating position within the second housing part 20. The second object 4B, as shown in FIG. 17, remains under the skin layer 6 at an angle to the first object 4A which corresponds to the horizontal angle WH.

Following this second or final placement procedure, both the cannula 10 and the tappet 32, as described above, are received completely in the second housing part 20. By manually sliding the first housing part 18 relative to the second housing part 20, the housing 16 can then be closed and safely stored or disposed of.

For safe disposal of all parts that have been contaminated during the placement procedures, in particular by particles of tissue and other dirt, the entire implantation device 2 can be designed as a single-use product that is disposed of as a whole. Alternatively, it is also possible to fasten the cannula 10 to the cannula holder 22 via a detachable connection and/or the tappet 32 to the first housing part 18 via a detachable connection, such as a plug connection, so that both parts can be replaced by new parts after the placement procedures have been completed. In addition, it is alternatively possible to design the cannula 10, in conjunction with at least parts of the cannula holder 22, as a single-use device, which can be fixed exchangeably to the rest of the implantation device 2.

FIGS. 18 to 20 show a further alternative embodiment of the housing 16 of the implantation device 2, in which the entire second housing part 20 is designed as a single-use device and is held exchangeably on the first housing part 18. The cannula 10 and the tappet 32 can also be disposed of with the second housing part 20, or replaced by new parts. In order to prevent inadvertent detachment of the second housing part 20 during operation, a safety coupling (not shown) is provided between the first housing part 18 and the second housing part 20, which coupling is only released or can only be released in the final operating position of the cannula 10 or the cannula holder 22. As a result, the second housing part 20 can be removed from the rest of the implantation device 2 after all the intended implantation procedures have been completed, as shown in FIG. 18.

In order to be able to reuse the rest of the implantation device 2, means are provided on the first housing part 18 for re-tensioning the mechanical energy store 30 or the rolled spring 28, which can be seen in particular in FIG. 19. These comprise a removable housing section 46 of the first housing part 18, which is connected to the rolled spring 28 via a tension member 48. This housing section 46 can be detached and pulled away from the rest of the first housing part 18 by pressing a safety catch 50, which is designed, for example, as a push button on the housing section 46, in order to reload the mechanical energy store 30 via the tension member 48 and the rolled spring 28, as shown in FIG. 19.

Next, the housing section 46 is reconnected to the remaining first housing part 18, so that a new second housing part 20 can then be attached to the first housing part 18. As can be seen from FIG. 20, this new second housing part 20 is, for positionally stable attachment to the first housing part 18, provided with both a new cannula 10 with new objects 4A, 4B to be implanted and a new tappet 32.

FIG. 21 shows a further embodiment of the implantation device 2, which has a functional sequence when implanting the objects 4A and 4B that substantially corresponds to the above-described embodiments of the implantation device 2. The embodiment according to FIG. 21 differs in that the return means for displacing the cannula 10 over the return stroke paths are actuated by manual application of force.

For this purpose, a sliding button 52 is provided on the first housing part 18, which, in the first insertion position, is fixed in a distal stop position of a guide 56 via a lock 54, which is formed, for example, by snap hooks and engagement elements cooperating with them. The sliding button 52 is connected to the cannula holder 22, on which, in accordance with the above-described embodiments of the implantation device 2, the cannula 10, into which the tappet 32 protrudes, is held.

After the cannula 10 has been inserted and slid into a first insertion position under the skin layer 6, the lock of the sliding button 52 on the first housing part 18 can be released by manual application of a force D and the sliding button 52 can be displaced by means of a manual sliding force 5 along the guide 56 into the first operating position as in FIG. 23. By this means, as shown, the first object 4A is deposited under the skin layer 6.

In preparation for the second placement procedure, the application of the force D to the sliding button 52 is ended, thereby restoring its lock 54 on the first housing part 18. The first housing part 18 together with the cannula holder 22 and the tappet 32 can then be slid in the distal direction with respect to the second housing part 20 into the second insertion position, in order to position the cannula 10 in the second implantation area under the skin layer 6, as shown in FIG. 24.

To place the second object 4B, the sliding button 52 is again subjected to the force D to release the lock 54, as shown in FIG. 25, and at the same time displaced into the second operating position, as shown in FIG. 26, by application of the sliding force S, whereby the second object 4B is deposited under the skin layer 6.

Irrespective of the respective embodiment of the implantation device 2, housing locking means 58 can be provided between the first housing part 18 and the second housing part 20, as shown by way of example in FIG. 27 using a locking hook 58A and a locking receptacle 58B which cooperates with said locking hook. Before the first operating position is reached, the locking hook 58A is in a locking position, in which it engages into the locking receptacle 58B and thereby fixes the first housing part 18 to the second housing part 20, as can be seen, for example, from FIGS. 2 and 22. This mutual fixing can prevent a troublesome relative movement during the insertion of the cannula 10 until the first operating position of the cannula holder 22 is reached. However, as soon as the first operating position is reached, the locking hook 58A disengages from the locking receptacle 58B, so that now a relative movement between the housing parts 18, 20 is possible. In addition, it can be provided that after completion of the final implantation procedure, the locking means 58 return to the locking position when the housing is closed as shown in FIG. 7, in order to again block a relative movement between the housing parts 18, 20 in this end position.

As illustrated above, the at least two objects 4A, 4B to be implanted can be pre-loaded in a ready-to-use state in the cannula 10 before the implantation device 2 is used. Alternatively, the implantation device 2 can also be designed as part of an implantation arrangement in which at least one object 4 to be implanted is initially present as a separate element which must be inserted into the cannula 10 before the implantation device is used. For this purpose, an insertion aid 60 can be provided which has, as shown for example in FIG. 28, a funnel-shaped part which can be attached to the distal tip 12 of the cannula 10 in order to be able to insert the relevant object 4 more easily into the cavity 8 of the cannula 10 by means of a handgrip 62.

In order in particular to be able to insert two or more objects 4A, 4B more easily into the cannula 10, these can alternatively or additionally be received in a sleeve, as can be seen in FIG. 29.

Irrespective of the respective embodiment, the implantation device 2 can be used to implant objects 4A, 4B with different functions. Particularly in the case of objects 4A, 4B for which the mode of operation depends on a suitable rotational position with respect to their longitudinal axis A relative to the skin layer 6, the cannula 10 or its cavity 8 can, as can be seen from FIGS. 30 and 31, have a cross-section which deviates from a circular shape and through which the correspondingly shaped object 4A, 4B can be guided and placed in a positionally stable manner with respect to the longitudinal axis A. For this purpose, the cross-section can, for example, be polygonal, oval, kidney-shaped, elliptical or the like.

It is noted that all of the elements and features of the various embodiments of the subject matter according to the invention described above are exchangeable or combinable with one another, provided that an exchange or a combination thereof is not ruled out for technical reasons.

1. An implantation device for positioning at least one object under a patient's skin, comprising:
a housing;
a cannula holder displaceable relative to the housing and on which a cannula with a distal tip and a cavity for receiving the at least one object to be implanted can be attached; and
a tappet which can be fixed relative to the housing and which has a distal end that is positionable in the cavity of the cannula,
wherein the cannula is displaceable relative to a housing outlet via a first return stroke path along the tappet, from a first insertion position to a first operating position, in order to deposit a first object received in the cavity,
wherein the cannula is displaceable together with the tappet from the first operating position into a second insertion position relative to the housing outlet and from the second insertion position via a second return stroke path along the tappet into a second operating position, in order to deposit a second object received in the cavity, and
wherein, for displacement into the second insertion position, the cannula holder can be fixed to a first housing part, which is fixedly connected to the distal end of the tappet and is held displaceably on a second housing part which forms the housing outlet.

2. The implantation device according to claim 1, wherein the first housing part is slidably mounted on the second housing part.

3. The implantation device according to claim 1, wherein the second housing part has anti-slip means on an outer side thereof.

4. The implantation device according to claim 1, wherein between the first housing part and the second housing part, housing locking means are provided, which fix the two housing parts onto each other until the first insertion position of the cannula is reached.

5. The implantation device according to claim 1, wherein return means are provided, by means of which the displacement of the cannula over at least one of the return stroke paths can be carried out automatically.

6. The implantation device according to claim 5, wherein the return means have a mechanical energy store, by means of which the cannula holder is displaceable.

7. The implantation device according to claim 5, wherein the return means have a gear mechanism, by which the cannula holder is displaceable.

8. The implantation device according to claim 5, wherein the return means have a damping device, by means of which a displacement speed during the displacement over at least one of the return stroke paths can be set.

9. The implantation device according to claim 5, wherein the return means have a trigger button, by means of which the displacement over at least one of the return stroke paths can be triggered.

10. The implantation device according to claim 9, wherein fixing means are provided on the trigger button and can be detachably connected to complementary fixing means of the cannula holder.

11. The implantation device according to claim 1, wherein the displacement over at least one of the return stroke paths can be carried out manually.

12. The implantation device according to claim 11, wherein, for manual displacement, a sliding button is provided, which is slidable on the housing and can be released from a lock by the application of force.

13. The implantation device according to claim 1, wherein in a final operating position the cannula is received completely within the housing.

14. The implantation device according to claim 13, wherein an end position safety device is provided, by means of which the cannula holder can be fixed in the final operating position relative to the housing.

15. The implantation device according to claim 13, wherein the tappet can be separated from the housing in the second operating position.

16. The implantation device according to claim 1, wherein the cannula and the tappet have a cross-section which deviates from a circular profile.

17. The implantation device according to claim 1, wherein a stop is provided at a distal end of the housing to limit the insertion depth.

18. The implantation device according to claim 17, wherein a contact surface bevelled at a specified insertion angle is formed on the stop.

19. The implantation device according to claim 1, wherein a viewing area is provided on the housing, through which a section of the cannula protruding from the housing outlet can be seen.

20. The implantation device according to claim 1, wherein the cannula is held exchangeably.

21. The implantation device according to claim 20, wherein the tappet is held exchangeably together with the cannula.

22. The implantation device according to claim 20, wherein the cannula holder is at least partially configured as a single-use device which can be fixed exchangeably on the rest of the device.

23. The implantation device according to claim 20, wherein the second housing part, which forms the housing outlet, is held exchangeably on the first housing part with the cannula.

24. The implantation device according to claim 20, wherein the exchangeable elements can be fixed on the rest of the device via a safety coupling, wherein the safety coupling is detachable in the final operating position.

25. The implantation device according to claim 20, wherein, for reuse of the implantation device with a new exchangeable element, the mechanical energy store can be pretensioned by manual relative movement of a housing section, wherein a safety catch is provided, which in an initial position blocks the relative movement of the housing section and can be displaced into a release position in which the relative movement is released.

26. The implantation device according to claim 20, wherein at least one object to be implanted is received in the cannula.

27. The implantation device according to claim 26, wherein the received object is disposed at the distal tip of the cannula.

28. An implantation arrangement comprising the implantation device according to claim 1 wherein at least one separate object to be implanted is also provided, which can be inserted into the cannula.

29. The implantation arrangement according to claim 28, wherein an insertion aid for inserting the at least one object into the cavity is provided, which can be attached to one end of the cannula.

30. The implantation arrangement according to claim 28, wherein the at least one object is received in a sleeve which can be inserted into the cannula.
